(19) United States
(12) Patent Application Publication
(10) Pub. No.: US 2026/0021102 A1
(43) Pub. Date: Jan. 22, 2026

(54) COMPOSITION AND METHOD FOR EXPEDITING DENTAL ANESTHESIA RECOVERY

(71) Applicant: David Drescher, Avon, CO (US)

(72) Inventor: David Drescher, Avon, CO (US)

(21) Appl. No.: 19/254,270

(22) Filed: Jun. 30, 2025

Related U.S. Application Data
(60) Provisional application No. 63/672,888, filed on Jul. 18, 2024.

(51) Int. Cl. A61K 31/522; A61K 31/05; A61K 31/167; A61K 31/375; A61K 31/445; A61K 31/4468; A61K 31/5517; A61K 36/21

(52) U.S. Cl. CPC A61K 31/522; A61K 31/05; A61K 31/167; A61K 31/375; A61K 31/445; A61K 31/4468; A61K 31/5517; A61K 36/21

(57) ABSTRACT

A dental composition for expediting recovery from dental anesthesia comprises Vitamin C (75-2,000 mg), caffeine (30-400 mg), and beet root extract (250-7,000 mg) in synergistic combination. The composition addresses prolonged numbness following dental procedures by employing multiple complementary mechanisms: Vitamin C counteracts anesthetic effects through neurochemical pathways, beet root extract enhances vascular delivery via increased blood flow, and caffeine accelerates metabolic clearance. Administered as capsules immediately after or during dental procedures, the composition reduces post-procedural numbness duration, prevents soft tissue injuries from inadvertent biting, restores normal speech and eating function sooner, and promotes wound healing. The invention provides a safe, effective solution to a long-standing problem in dental anesthesia management.

### CROSS REFERENCE TO RELATED APPLICATIONS

This application claims the benefit of U.S. Provisional Patent Application No. 63/672,888, filed Jul. 18, 2024, which is hereby incorporated by reference in its entirety.

### FIELD OF THE INVENTION

The present invention relates generally to the field of dentistry and pharmaceutical compositions. More particularly, the present invention relates to compositions and methods for expediting recovery from dental anesthesia administered during dental procedures.

### BACKGROUND OF THE INVENTION

Dental procedures have historically required the use of anesthetic agents to manage pain and discomfort during treatment. Local anesthetics such as lidocaine, mepivacaine, bupivacaine, prilocaine, and articaine are commonly injected to numb specific areas of the mouth. Additionally, sedation anesthetics such as nitrous oxide, and oral anesthetics including midazolam, propofol, and fentanyl may be employed for more complex procedures or anxious patients.

While these anesthetic agents are essential for patient comfort during dental procedures, they present significant problems in the post-procedural period. Patients consistently complain about prolonged numbness following completion of their dental procedures. The anesthetic effect typically lasts 2-3 hours on average, while most routine dental procedures are completed within 30 minutes to one hour. This extended duration of numbness creates several problematic scenarios for patients.

One of the most concerning risks associated with prolonged dental anesthesia is the potential for soft tissue injury. Patients frequently bite their tongue, cheeks, or lips without realizing it due to the lack of sensation, leading to painful injuries that can become infected or take extended time to heal. Additionally, the numbness prevents normal eating and drinking, as patients cannot properly control their jaw and tongue movements.

The social and professional impacts of prolonged dental anesthesia are equally problematic. Speech becomes impaired due to the inability to properly control the tongue and lips, causing patients to sound as though they have speech impediments. This can significantly impact work productivity, particularly for individuals whose jobs require clear communication. The cosmetic effects, such as drooping lips and distorted facial expressions, can cause embarrassment and social anxiety.

Despite these well-recognized problems, there has been little available to patients to accelerate recovery from dental anesthesia. The standard recommendation has been to exercise extreme caution and simply wait for the anesthetic to be naturally metabolized and eliminated from the body.

While the prior art discloses various compositions containing some similar ingredients to those useful for the present invention, these compositions are directed to entirely different purposes and fail to address the specific problems associated with dental anesthesia recovery.

For example, U.S. patent application Ser. No. 15/299,887 to Depta filed on Oct. 10, 2016, hereby incorporated by reference, discloses a composition comprising red spinach extract, red beet extract, and aronia berry extract intended to increase athletic energy and endurance and enhance post-workout muscle recovery. However, this reference is directed to athletic performance enhancement rather than addressing the specific challenges of dental anesthesia recovery.

Similarly, U.S. Pat. No. 10,624,921 to Green dated Apr. 21, 2020, hereby incorporated by reference, describes compositions for improving nitric oxide levels using dietary nitrate derived from potassium nitrate, beet root, and nitrate-rich leafy greens. While this reference discusses cardiovascular benefits including lowering blood pressure and improving endothelium function, it fails to address the neurological effects of anesthetic agents or provide solutions for expediting anesthetic recovery.

U.S. Pat. No. 11,026,986 to Noguchi dated Jun. 8, 2021, hereby incorporated by reference, discloses a beetroot-containing food composition in the form of soup, sauce, or condiment that prevents blood pressure increase while masking the taste of nitrate-containing vegetables. This reference is focused on dietary applications and blood pressure management, not anesthetic recovery.

Canada Patent No. CA3050823C to Tarnopolsky describes a weight management composition comprising beetroot extract along with other agents like green tea extract, green coffee bean extract, and forskolin. This reference is directed to weight loss and mitochondrial enhancement, which are unrelated to the problems of prolonged dental anesthesia.

Other patent documents, including U.S. Patent Application No. 2016/0303177 to Bailey and U.S. Patent Application No. 2023/0181487 to Crawford, similarly focus on general nutritional supplementation rather than addressing the specific medical need for accelerated anesthetic recovery.

None of the such known prior art compositions are formulated specifically to counteract the effects of dental anesthesia or provide the rapid onset of action necessary to meaningfully reduce the duration of post-procedural numbness. Furthermore, none of the prior art recognizes or addresses the specific combination of physiological mechanisms necessary to effectively antagonize anesthetic agents and restore normal sensation to anesthetized tissues.

Accordingly, there remains a long-felt but unmet need for a composition specifically designed to expedite recovery from dental anesthesia. Such a composition should be capable of safely and effectively reducing the duration of numbness following dental procedures, thereby preventing soft tissue injuries, restoring normal speech and eating function, and allowing patients to return to their normal activities more quickly. The composition should work through multiple complementary mechanisms to ensure rapid and reliable reversal of anesthetic effects while promoting healing of any procedural trauma.

### SUMMARY OF THE INVENTION

The present invention provides a dental composition specifically formulated to expedite recovery from dental anesthesia and a method for administering the composition to achieve rapid restoration of normal sensation following dental procedures. The invention addresses the long-standing problem of prolonged numbness that persists for 2-3 hours after routine dental procedures, which typically last only 30 minutes to one hour.

In one embodiment, the dental composition comprises three synergistic active ingredients: Vitamin C in an amount from about 75 mg to about 2,000 mg, caffeine in an amount from about 30 mg to about 400 mg, and beet root extract in an amount from about 250 mg to about 7,000 mg. The preferred formulation contains approximately 1,000 mg of Vitamin C, 250 mg of beet root extract, and 45 mg of caffeine.

The composition works through multiple complementary mechanisms to achieve rapid anesthetic recovery. Vitamin C serves as the primary antagonistic agent that counteracts the effect of anesthetic agents by opposing analgesic action through neurochemical pathways. Beet root extract functions as a vascular enhancement component that opens up the vasculature to speed up the anesthetic clearance process by increasing blood flow to the treated area. Caffeine provides metabolic acceleration that bumps up metabolism to speed up the anesthetic clearance process while also accelerating the action of Vitamin C on the affected area through increased metabolic activity.

The synergistic interaction of these three components enables quicker delivery of active ingredients via the bloodstream and provides enhanced efficacy beyond what could be achieved by any single component acting alone. Clinical studies demonstrate the effectiveness of Vitamin C in accelerating the wearing off of anesthetic effects, while the composition as a whole is known to accelerate the wearing off of anesthetic effects.

In another embodiment, the invention provides a method of expediting recovery from dental anesthesia comprising administering the dental composition to a person who has received dental anesthesia. The administration protocol is tailored to the procedure duration: for short procedures lasting approximately one hour, the composition is ingested immediately after administration of anesthetic, while for extended procedures lasting approximately two hours, the composition is administered during the procedure.

The composition is effective against various types of anesthetics including local anesthetics such as lidocaine, mepivacaine, bupivacaine, prilocaine, and articaine, sedation anesthetics such as nitrous oxide, and oral anesthetics including midazolam, propofol, and fentanyl. This broad spectrum effectiveness makes the composition universally applicable across different dental practices and anesthetic preferences.

The invention provides multiple clinical benefits including expedited recovery from numbness, injury prevention by reducing the risk of soft tissue damage from inadvertent biting of the tongue, cheeks, or lips, functional restoration enabling normal eating, drinking, and speech patterns sooner, and healing promotion through the secondary benefit of enhanced wound healing provided by Vitamin C supplementation. The composition addresses the social and professional impacts of prolonged anesthesia, including speech impairment and cosmetic effects that can cause embarrassment and negatively impact work productivity.

In a preferred embodiment, the dental composition is formulated as a standard gelatin capsule containing the specified ingredient ratios, with a dosage form of one to two capsules depending on the complexity and duration of the dental procedure. The composition may include pharmaceutically acceptable excipients such as binding agents, suspending agents, buffering agents, and preservatives.

The composition exhibits a minimal risk profile with clinical assessment showing minimal risks while providing significant benefits to patients. Patients with known allergies or sensitivities to any ingredient are excluded from treatment to ensure safety.

For pediatric applications, the composition may exclude caffeine while maintaining the core therapeutic benefits of Vitamin C and beet root extract for anesthetic recovery.

The invention also encompasses compositions that are in the absence of various other ingredients commonly found in nutritional supplements, including forskolin, green tea extract, green coffee bean extract, coenzyme Q10, alpha lipoic acid, vitamin E, potassium nitrate, pomegranate fruit extract, thiamin, folate, vitamin B12, potassium, Rhodiola extract, French pine extract, Pulsatilla vulgaris extract, ginger extract, yohimbe extract, ginseng extract, Coleus forskolii extract, and arginine nitrate.

The present invention represents a significant advancement in dental anesthesia management by providing the first composition specifically designed to address the technical problem of prolonged post-procedural numbness through a scientifically-based, multi-mechanism approach that ensures rapid and reliable reversal of anesthetic effects while promoting healing.

### BRIEF DESCRIPTION OF THE FIGURES

The accompanying drawings, which are incorporated herein and form a part of the specification, illustrate the presently preferred embodiments of the invention and, together with the general description given above and the detailed description of the preferred embodiments given below, serve to explain the principles of the invention.

FIG. 1 illustrates a chart showing the contents and dosages of the active components of the dental composition of the present invention, specifically depicting Vitamin C, Beet Root Extract, and Caffeine in the preferred formulation.

FIG. 2 is a flow chart illustrating the steps and actions that take place with the administration of the dental composition of the present invention, showing the sequence from post-dental procedure capsule administration through the synergistic mechanisms of action to the resulting reduction in numbness time and promotion of healing.

FIG. 3 illustrates a conventional dental procedure scenario, showing in the upper portion a dental practitioner administering anesthetic to a patient, and in the lower portion the patient experiencing the problematic effects of prolonged numbness and discomfort following completion of the dental procedure.

FIG. 4 illustrates the therapeutic intervention and outcome using the dental composition of the present invention, showing in the upper portion the patient consuming the dental composition immediately following the procedure, and in the lower portion the patient experiencing alleviated numbness and restored normal function.

### DETAILED DESCRIPTION

The preferred embodiment of the present invention relates generally to the field of dental anesthesia recovery compositions and methods for expediting the return of normal sensation following dental procedures. More particularly, the present invention addresses the technical problem of prolonged numbness that occurs after administration of local, sedation, or oral anesthetics during dental treatments, providing a synergistic composition specifically formulated for expedited anesthetic recovery.

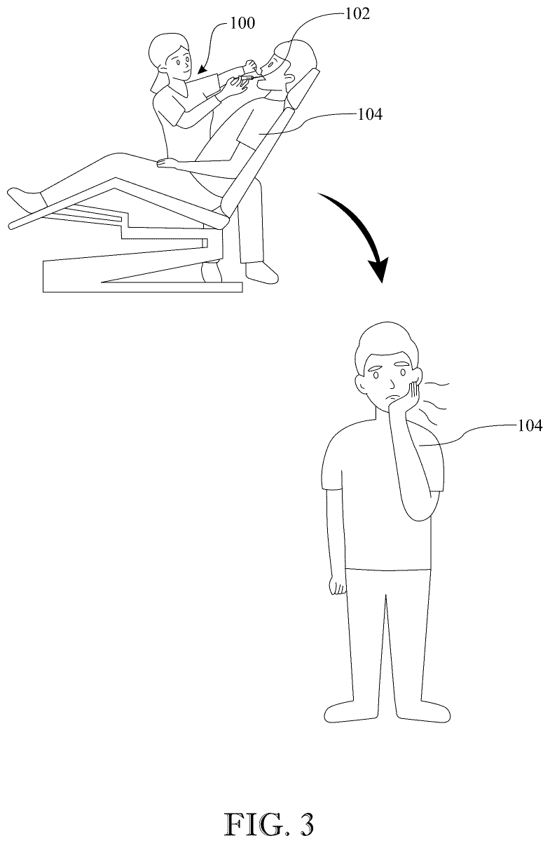

As illustrated in FIG. 3, conventional dental procedures involve the administration of anesthetic agents 102 by a dental practitioner 100 to a patient 104 to ensure comfort during treatment. However, as further shown in FIG. 3, patients 104 typically experience extended periods of numbness and discomfort following completion of the dental procedure, often lasting 2-3 hours beyond the actual treatment time. This prolonged anesthetic effect creates significant problems including risk of soft tissue injury, impaired speech and eating function, and general patient dissatisfaction.

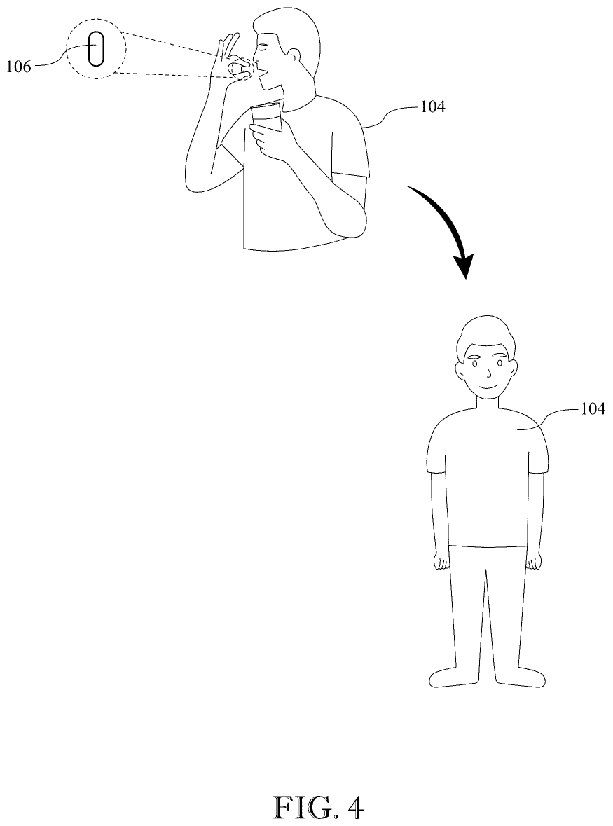

The present invention in its preferred embodiment provides a novel solution through a carefully formulated composition that works synergistically to counteract anesthetic effects and restore normal sensation. As demonstrated in FIG. 4, administration of the dental composition 106 to the patient 104 immediately following or during the dental procedure results in significantly reduced numbness duration and enhanced recovery, allowing patients to return to normal function more rapidly.

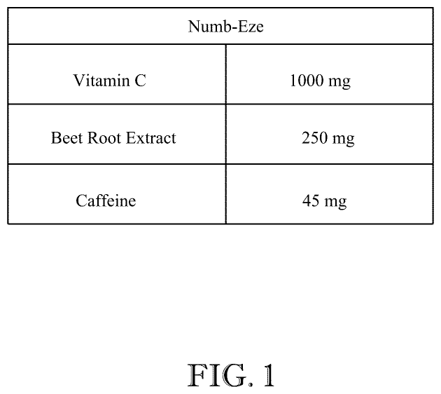

The dental composition of the preferred embodiment comprises three primary active ingredients working in synergistic combination to achieve expedited anesthetic recovery. As shown in FIG. 1, the preferred formulation contains Vitamin C (1000 mg), Beet Root Extract (250 mg), and Caffeine (45 mg), each component contributing distinct but complementary mechanisms of action for optimal therapeutic effect.

Vitamin C serves as the primary antagonistic agent in the composition, with a dosage range of 75 mg to 2,000 mg, preferably from 750 mg to 1,100 mg, and most preferably about 1,000 mg as illustrated in FIG. 1. The primary function of Vitamin C is to counteract the effect of anesthetic agents through its ability to oppose analgesic action via specific neurochemical pathways. Clinical studies demonstrate Vitamin C's effectiveness in accelerating the wearing off of anesthetic effects, making it the foundational component of the present composition. Additionally, Vitamin C provides a secondary benefit of promoting healing and wound recovery as clinically demonstrated, enhancing the overall therapeutic value of the composition beyond mere anesthetic reversal.

Beet Root Extract functions as the vascular enhancement component of the composition, with a dosage range of 250 mg to 7,000 mg, with a preferred embodiment of about 250 mg as shown in FIG. 1. The primary function of Beet Root Extract is to open up the vasculature to speed up the anesthetic clearance process through increased blood flow to the treated area via vasodilation. This vascular enhancement plays a critical role in enabling quicker delivery of the active components via the bloodstream, thereby facilitating the rapid distribution and action of the other compositional elements, particularly Vitamin C, to the anesthetized tissues.

Caffeine serves as the metabolic acceleration component, with a dosage range of 30 mg to 400 mg, preferably from 42 mg to 100 mg, and most preferably about 45 mg as depicted in FIG. 1. The primary function of caffeine is to enhance metabolism to speed up the anesthetic clearance process, working through a mechanism that opposes analgesic action similar to Vitamin C. Caffeine's enhancement role involves accelerating the action of Vitamin C on the affected area through increased metabolic activity, creating a synergistic effect that amplifies the overall therapeutic benefit of the composition beyond what could be achieved by any single component alone.

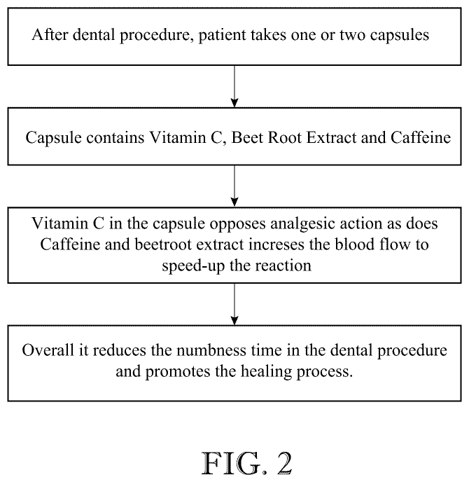

The dental composition of the present invention achieves superior therapeutic results through the synergistic interaction of its three active components, each contributing distinct but complementary mechanisms that work in concert to expedite anesthetic recovery. As illustrated in FIG. 2, this synergistic approach provides enhanced efficacy beyond what could be achieved by any single component acting alone.

In a preferred embodiment, the composition employs dual antagonistic action wherein both Vitamin C and caffeine oppose analgesic effects through different but complementary pathways. Vitamin C functions as the primary neurochemical antagonist, directly counteracting the anesthetic agents at the receptor level, while caffeine provides metabolic enhancement that accelerates the overall clearance process. This dual mechanism ensures multiple pathways for anesthetic reversal, providing redundancy and enhanced reliability of therapeutic effect.

The vascular enhancement provided by beet root extract serves as the critical delivery mechanism that facilitates rapid distribution of the active components throughout the anesthetized tissues. By opening up the vasculature and increasing blood flow to the treated area, the beet root extract enables quicker delivery of both Vitamin C and caffeine via the bloodstream, ensuring that these antagonistic agents reach their target sites rapidly and in sufficient concentration to achieve therapeutic effect.

In an embodiment of the invention, caffeine provides metabolic acceleration that enhances overall metabolic clearance of anesthetic agents while simultaneously accelerating the action of Vitamin C on the affected area through increased metabolic activity. This creates a multiplicative effect wherein the caffeine not only contributes its own anesthetic-opposing properties but also amplifies the effectiveness of the Vitamin C component.

The clinical efficacy of the dental composition has been demonstrated through controlled clinical protocols specifically designed to evaluate its effectiveness in reducing prolonged anesthesia duration. In a preferred embodiment, the primary objective achievement has been demonstrated through clinical protocols that show clear efficacy in reducing prolonged anesthesia duration when compared to subjects receiving no treatment.

The clinical study design employed randomized controlled methodology showing superior results versus no treatment controls. As detailed in FIG. 2, subjects receiving the dental composition demonstrated measurably shorter duration of numbness and faster return to normal sensation compared to control subjects who received no intervention.

In an embodiment of the invention, measurable outcomes demonstrate that subjects emerge from anesthesia sooner, allowing normal routine function to resume more rapidly following dental procedures. The clinical assessment reveals minimal risks associated with the composition while providing significant benefits to patients, establishing a favorable risk-benefit profile that supports the therapeutic utility of the invention.

In a preferred embodiment, the dental composition is formulated as a delivery system utilizing standard gelatin capsule format, as illustrated in FIG. 1. This capsule delivery method provides convenient oral administration while ensuring accurate dosing and optimal bioavailability of the active ingredients.

The dosage form comprises one to two capsules containing the specified ingredient ratios as shown in FIG. 1. In the preferred embodiment, a single capsule contains Vitamin C 1000 mg, Beet Root Extract 250 mg, and Caffeine 45 mg, with the option for patients to take two capsules for enhanced effect based on the complexity and duration of their dental procedure.

In an embodiment of the invention, the composition may include pharmaceutically acceptable excipients such as binding agents, suspending agents, buffering agents, and preservatives as detailed in the provisional application. These excipients serve to maintain the stability and integrity of the active ingredients while facilitating proper capsule formation and dissolution upon oral administration.

The administration protocol is specifically tailored to the duration and complexity of the dental procedure to optimize therapeutic benefit. In a preferred embodiment, for short procedures lasting approximately one hour, the composition is ingested immediately after administration of anesthetic, as illustrated in the flow chart of FIG. 2.

For extended procedures lasting approximately two hours, an embodiment of the invention provides for administration during the procedure rather than after completion. This timing ensures that the active components begin their anesthetic-opposing action as the dental procedure concludes, providing seamless transition from anesthetized state to normal sensation.

In a preferred embodiment, the composition is effective against multiple types of local anesthetics including lidocaine, mepivacaine, bupivacaine, prilocaine, and articaine. This broad spectrum effectiveness makes the composition universally applicable across different dental practices and anesthetic preferences, providing consistent therapeutic benefit regardless of the specific anesthetic agent employed during the dental procedure.

The administration protocol as depicted in FIG. 4 demonstrates the therapeutic outcome wherein patient 104 experiences alleviated numbness and restored normal function following oral consumption of the dental composition 106, providing visual confirmation of the invention's clinical effectiveness in real-world application.

The dental composition of the present invention finds its primary application in local anesthetic recovery, being specifically designed for dental procedure aftermath as illustrated in FIG. 4. In a preferred embodiment, the composition addresses the well-recognized problem of prolonged numbness following routine dental procedures, which typically lasts 2-3 hours while most procedures are completed within 30 minutes to one hour.

In an embodiment of the invention, the composition has potential extended uses that are applicable for dermatological procedures requiring local anesthesia. The versatility of the composition's mechanism of action makes it suitable for any medical or cosmetic procedure where local anesthetic agents are employed and rapid recovery of normal sensation is desired.

The preferred embodiment demonstrates universal compatibility, being effective with various anesthetic agents used in dental practice. As detailed in the provisional application and shown in FIG. 2, the composition is effective against local anesthetics including lidocaine, mepivacaine, bupivacaine, prilocaine, and articaine, providing broad spectrum applicability across different dental practices and anesthetic preferences.

In a preferred embodiment, the composition provides expedited recovery that is known to accelerate the wearing off of anesthetic effects. Clinical studies demonstrate measurable reduction in the duration of post-procedural numbness, allowing patients to return to normal activities significantly sooner than would occur with natural anesthetic metabolism alone.

A critical benefit of the invention is injury prevention, which reduces the risk of soft tissue damage from inadvertent biting. As documented in the clinical rationale, patients frequently bite their tongue, cheeks, or lips without realizing it due to lack of sensation, leading to painful injuries that can become infected or take extended time to heal. The rapid restoration of sensation provided by the composition significantly mitigates this risk.

In an embodiment of the invention, functional restoration enables normal eating, drinking, and speech patterns sooner following dental procedures. The composition addresses the social and professional impacts of prolonged anesthesia, including speech impairment and the cosmetic effects of drooping lips that can cause embarrassment and negatively impact work productivity.

The preferred embodiment exhibits a minimal risk profile, with clinical assessment showing minimal risks while providing significant benefits to patients. Patients with known allergies or sensitivities to any ingredient are excluded from treatment, ensuring safety while maximizing therapeutic benefit.

An additional benefit of the invention is healing promotion through the secondary benefit of enhanced wound healing provided by Vitamin C supplementation. As demonstrated in clinical studies, Vitamin C is clinically shown to promote healing and wound recovery, providing value beyond mere anesthetic reversal.

The preferred embodiment utilizes a standard adult formulation comprising the primary composition of Vitamin C 1000 mg, Beet Root Extract 250-450 mg, and Caffeine 45 mg as illustrated in FIG. 1. This specific ratio has been optimized through clinical testing to provide maximum therapeutic benefit while maintaining safety and tolerability.

In an embodiment of the invention, alternative ratios provide flexible dosing within the specified ranges based on procedure complexity and patient factors. The composition may be adjusted within the disclosed ranges of Vitamin C 75 mg to 2,000 mg, Beet Root Extract 250 mg to 7,000 mg, and Caffeine 30 mg to 400 mg to accommodate varying degrees of anesthetic intensity and individual patient response.

For pediatric applications, an embodiment of the invention provides formulation considerations that may exclude caffeine for pediatric applications. This modification recognizes the particular sensitivities of younger patients while maintaining the core therapeutic benefits of Vitamin C and Beet Root Extract for anesthetic recovery.

The preferred embodiment includes appropriate contraindications requiring exclusion of patients with known allergies or sensitivities to any ingredient. This safety measure ensures that the composition is administered only to patients who can safely benefit from its therapeutic effects, maintaining the favorable risk-benefit profile established in clinical studies.

The present invention provides a novel and synergistic dental composition specifically formulated to address the long-standing problem of prolonged numbness following dental anesthesia. Through the carefully balanced combination of Vitamin C, Beet Root Extract, and Caffeine, as illustrated in FIGS. 1 and 2, the composition works through multiple complementary mechanisms to expedite anesthetic recovery while promoting healing. Vitamin C serves as the primary antagonistic agent, directly counteracting anesthetic effects through neurochemical pathways, while Beet Root Extract enhances vascular delivery through increased blood flow, and Caffeine accelerates metabolic clearance of anesthetic agents. The synergistic interaction of these components, as demonstrated through clinical protocols and comparative studies, provides patients with measurably shorter duration of post-procedural numbness, reduced risk of soft tissue injury, restored normal speech and eating function, and enhanced wound healing. The composition's broad spectrum effectiveness against various local anesthetics, combined with its minimal risk profile and flexible dosing options for different patient populations, establishes it as a significant advancement in dental anesthesia management. As shown in FIG. 4, patients who receive the dental composition experience alleviated numbness and restored normal function, demonstrating the practical clinical value of this innovative therapeutic approach to one of dentistry's most persistent post-procedural challenges.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

I claim:

1. A dental composition for expediting recovery from dental anesthesia comprising:
from 75 mg to 2,000 mg of vitamin C;
from 30 mg to 400 mg of caffeine; and
from 250 mg to 7,000 mg of beet root extract.

2. The dental composition of claim 1, wherein the vitamin C is present in an amount from 750 mg to 1,100 mg.

3. The dental composition of claim 2, wherein the vitamin C is present in an amount of 1,000 mg.

4. The dental composition of claim 1, wherein the caffeine is present in an amount from 42 mg to 100 mg.

5. The dental composition of claim 4, wherein the caffeine is present in an amount of 45 mg.

6. The dental composition of claim 1, wherein the beet root extract is present in an amount of 250 mg.

7. The dental composition of claim 1, comprising 1,000 mg of vitamin C, 250 mg of beet root extract, and 45 mg of caffeine.

8. The dental composition of claim 1, wherein the composition is formulated as a capsule.

9. The dental composition of claim 8, wherein the capsule is a gelatin capsule.

10. The dental composition of claim 1, wherein the dental anesthesia comprises a local anesthetic selected from the group consisting of lidocaine, mepivacaine, bupivacaine, prilocaine, and articaine.

11. The dental composition of claim 1, wherein the dental anesthesia comprises a sedation anesthetic selected from the group consisting of nitrous oxide.

12. The dental composition of claim 1, wherein the dental anesthesia comprises an oral anesthetic selected from the group consisting of midazolam, propofol, and fentanyl.

13. The dental composition of claim 1, wherein the composition consists essentially of vitamin C, caffeine, and beet root extract in the specified amounts.

14. The dental composition of claim 1, wherein the composition is in the absence of forskolin, green tea extract, green coffee bean extract, coenzyme Q10, alpha lipoic acid, vitamin E, potassium nitrate, pomegranate fruit extract, thiamin, folate, vitamin B12, potassium, Rhodiola extract, French pine extract, Pulsatilla vulgaris extract, ginger extract, yohimbe extract, ginseng extract, Coleus forskolii extract, and arginine nitrate.

15. The dental composition of claim 1, wherein the vitamin C counteracts the effect of anesthetic agents through opposing analgesic action.

16. The dental composition of claim 1, wherein the beet root extract increases blood flow to facilitate anesthetic clearance.

17. The dental composition of claim 1, wherein the caffeine enhances metabolism to accelerate anesthetic clearance.

18. A method of expediting recovery from dental anesthesia comprising:
administering to a person who has received dental anesthesia, a composition comprising:
from 75 mg to 2,000 mg of vitamin C;
from 30 mg to 400 mg of caffeine; and
from 250 mg to 7,000 mg of beet root extract.

19. The method of claim 18, wherein the composition is administered immediately after administration of the dental anesthesia.

20. The method of claim 18, wherein the composition is administered during a dental procedure requiring the dental anesthesia.

21. The method of claim 18, wherein the composition is administered for procedures lasting approximately one hour, and the composition is ingested immediately after administration of anesthetic.

22. The method of claim 18, wherein the composition is administered for procedures lasting approximately two hours, and the composition is administered during the procedure.

23. The method of claim 18, wherein the dental anesthesia is a local anesthetic comprising lidocaine.

24. The method of claim 18, wherein the composition accelerates the wearing off of anesthetic effects.

25. The method of claim 18, wherein the composition reduces the duration of post-procedural numbness.

26. The method of claim 18, wherein the composition reduces the risk of soft tissue injury from inadvertent biting.

27. The method of claim 18, wherein the composition promotes healing of procedural trauma through vitamin C supplementation.

28. The method of claim 18, wherein the person is an adult and the composition comprises about 1,000 mg of vitamin C, about 250 mg of beet root extract, and about 45 mg of caffeine.

29. The method of claim 18, wherein the person is a pediatric patient and the composition excludes caffeine.

30. The method of claim 18, wherein the composition is administered in a dosage form of one to two capsules.
